(19) United States
(12) Patent Application Publication
(10) Pub. No.: US 2011/0270275 A1
(43) Pub. Date: Nov. 3, 2011

(54) METHODS AND DEVICES FOR LIGATING TUBULAR STRUCTURES

(75) Inventor: Stephen D. Cassivi, Rochester, MN (US)

(73) Assignee: Mayo Foundation for Medical Education and Research, Rochester, MN (US)

(21) Appl. No.: 13/062,904

(22) PCT Filed: Sep. 8, 2009
(86) PCT No.: PCT/US09/56168

§ 371 Date: May 20, 2011

Related U.S. Application Data
(60) Provisional application No. 61/095,119, filed on Sep. 8, 2008.

(51) Int. Cl. A61B 17/10

(52) U.S. Cl. 606/139

(57) ABSTRACT

The devices and methods described herein may be used to ligate tissue, e.g., tubular structures where access is limited by using a sheath to deliver one or more sutures that can be deflected such that the end of the sheath forms a curve.

### RELATED APPLICATION

The present application claims the benefit under 35 U.S.C. §119(e) of U.S. Provisional Patent Application No. 61/095,119, filed on Sep. 8, 2008 and titled METHODS AND DEVICES FOR LIGATING TUBULAR STRUCTURES, which is hereby incorporated by reference in its entirety.

In a wide variety of applications, for example, ligating a tubular structure (e.g., artery, vein, bile duct, thoracic duct, cystic duct, ureter, fallopian tubes, etc.) may be desired. Ligating of tubular structures may be performed using, for example, sutures, clips, clamps, etc. In some circumstances, the tubular structure to be ligated may be fragile and/or in a difficult to access location for conventional ligating options, the ligation of which being technically challenging. Improved methods and devices for ligating a tubular structure are sought.

Ligation of, for example, a pulmonary artery during thorascopic procedures (e.g., video-assisted thoracic surgery (VATS)) may be technically challenging due to, for example, the pulmonary artery being uniquely fragile and the conventional devices used to perform the ligating being bulky and rigid.

### SUMMARY

Devices and methods for ligating tissue are described herein. The ligation devices may include a sheath, a lumen, and a suture. The sheath may include a distal end, a proximal end, and a steerable portion proximate the distal end, wherein a longitudinal axis extends through the sheath between the distal end and proximal end.

In some embodiments, the ligation devices described herein include a sheath having a distal end and a proximal end, wherein a longitudinal axis extends through the sheath between the proximal end and the distal end; a lumen extending through the sheath towards the distal end of the sheath, wherein the lumen is generally aligned with the longitudinal axis, and wherein the lumen has a port proximate the distal end of the sheath, wherein the port opens into the lumen; and a suture located within the lumen, wherein the suture can be advanced out of the lumen through the port; wherein the sheath includes a steerable portion proximate the distal end, wherein the steerable portion can be deflected such that the steerable portion forms a curve proximate the distal end of the sheath, and wherein the port is located within the steerable portion of the sheath.

In some embodiments of the ligation devices, the steerable portion of the sheath is flattened such that the steerable portion of the sheath has a cross-sectional profile that includes a major dimension and a minor dimension.

In some embodiments of the ligation devices, the suture may be a hollow suture that contains a stiffening element located therein.

In some embodiments of the ligation devices, the ligation device further includes a second lumen extending through the sheath towards the distal end of the sheath, wherein the second lumen is generally aligned with the longitudinal axis, and wherein the second lumen has a second port proximate the distal end of the sheath, wherein the second port opens into the second lumen, and further wherein the second port is located in the steerable portion of the sheath; a second suture located within the second lumen, wherein the second suture can be advanced out of the second lumen through the second port. In some embodiments, the second suture may be a second hollow suture.

In some embodiments of the ligation devices, a suture fastener may be included.

In some embodiments of the ligation methods described herein, the method may include providing a ligation device as described herein; deflecting the steerable portion of the sheath to form a curve proximate the distal end of the sheath; advancing the suture out of the lumen through the port on the distal end of the sheath; removing the sheath from the suture; and ligating a tubular structure to be ligated with the suture.

In some embodiments, the ligation device used in the method includes a second lumen extending through the sheath towards the distal end of the sheath, wherein the second lumen is generally aligned with the longitudinal axis, and wherein the second lumen has a second port proximate the distal end of the sheath, wherein the second port opens into the second lumen, and further wherein the second port is located in the steerable portion of the sheath; a second suture located within the second lumen, wherein the second suture can be advanced out of the second lumen through the second port. The method in such embodiments includes advancing the second suture out of the second lumen through the second port on the distal end of the sheath; removing the sheath from the second suture; and ligating the tubular structure to be ligated with the second suture.

In some embodiments of the methods described herein, each suture optionally includes a hollow suture that contains a stiffening element located therein and the method further includes removing each stiffening element from each hollow suture.

The above summary is not intended to describe each embodiment or every implementation of the devices and methods described herein. Rather, a more complete understanding of the devices and methods described herein will become apparent and appreciated by reference to the following Description of Illustrative Embodiments and claims in view of the accompanying figures.

### BRIEF DESCRIPTIONS OF THE FIGURES

FIG. 1 depicts a portion of one embodiment of a ligation device as described herein.

FIG. 2(a) depicts a cross-sectional profile of a steerable portion of a sheath that includes two lumens.

FIG. 2(b) depicts a cross-sectional profile of a steerable portion of a sheath that includes one lumen.

FIG. 3 depicts another embodiment of a ligation device in which a steerable portion of the ligation device is being deflected around a tubular structure to be ligated.

FIG. 4(a) depicts a portion of another embodiment of a ligation device in which a steerable portion of the ligation device is deflected around a tubular structure to be ligated along with a grasping device which may be used to grasp the sutures and/or the stiffening elements extending out of the ligation device.

FIG. 4(b) depicts removal of the ligation device of FIG. 4(a).

FIG. 4(c) depicts the two sutures of FIGS. 4(a) and 4(b) in place around the tubular structure after removal of the sheath.

FIG. 4(d) depicts use of the sutures of FIGS. 4(a)-4(c) to ligate the tubular structure by advancing suture fasteners toward the tubular structure.

### DESCRIPTION OF ILLUSTRATIVE EMBODIMENTS

In the following description of illustrative embodiments, reference is made to the accompanying figures which form a part hereof, and in which are shown, by way of illustration, specific embodiments in which the devices and/or methods may be practiced. It is to be understood that other embodiments may be utilized and structural changes may be made without departing from the scope of the present invention.

The devices and methods described herein may be used to ligate tissue, e.g., tubular structures where access is limited by using a sheath to deliver one or more sutures that can be deflected such that the end of the sheath forms a curve.

A sheath, according to the present disclosure, may be constructed from one or more of a wide variety of suitable materials. Some potentially suitable sheath materials may include, e.g., but are not limited to, those mentioned in U.S. Pat. No. 5,195,968 (Lundquist et al.), U.S. Pat. No. 6,610,058 (Flores), and U.S. Pat. No. 7,037,290 (Gardeski et al.). One having ordinary skill in the art will recognize other suitable sheath materials.

In one or more embodiments, the sheath is adapted to be advanced through an elongate passage for use in a thorascopic procedure, although it should be understood that the ligation devices described herein may be delivered to the tissue to be ligated using any suitable technique, e.g., open surgery, laparoscopic surgery, endoscopic surgery (e.g., Natural Orifice Translumenal Endoscopic Surgery (NOTES)), remote surgery (using, e.g., robotic systems); etc.

The devices and methods of the present disclosure may provide various advantages, such as potentially reducing likelihood of inadvertent damage to a tubular structure (e.g., artery) to be ligated and/or reducing technical difficulty of ligation.

A ligation device of the present disclosure includes a lumen in the sheath. The lumen may extend through the sheath towards the distal end of the sheath. The lumen may generally be aligned with the longitudinal axis extending through the sheath between the proximal end of the sheath and the distal end of the sheath.

The lumen may include a port proximate the distal end of the sheath. The port may open into the lumen.

In one or more embodiments, the lumen may extend from the distal end of the sheath to the proximal end of the sheath. Alternatively, in some embodiments, the lumen does not extend to the proximal end of the sheath. The lumen may terminate at a location between the distal end of the sheath and the proximal end of the sheath.

A lumen may be adapted to allow advancement of a suture therethrough and/or for advancement of other instruments. A suitable size for the lumen diameter is such that a suture and/or other instrument may be advanced therethrough.

A ligation device of the present disclosure may include a suture. The suture may be located within the lumen. The suture may be advanced through the lumen and the port. The suture may be advanced out of the lumen through the port. Thus, the suture may be adapted to pass through the lumen and the port.

Within the present disclosure, any suitable suture may be used. In one or more embodiments, a suture may be a hollow suture. A suture, according to the present disclosure, may be constructed from one or more of a wide variety of suitable materials. Some potentially suitable suture materials may include, e.g., but are not limited to, those mentioned in PCT Application Publication No. WO 2008/036408 (Friedman et al.). One having ordinary skill in the art will recognize other suitable suture materials.

In one or more embodiments of the present disclosure, the sheath includes a steerable portion proximate the distal end. The steerable portion may be deflected such that the steerable portion forms a curve proximate the distal end of the sheath. In one or more embodiments, the port is located within the steerable portion of the sheath.

Examples of sheaths having steerable portions may be found in, for example, U.S. Pat. No. 5,195,968 (Lundquist et al.), U.S. Pat. No. 6,610,058 (Flores), and U.S. Pat. No. 7,037,290 (Gardeski et al.). An example of a steering mechanism may be found in U.S. Pat. No. 5,195,968 (Lundquist et al.).

Within the present disclosure, the steerable portion of the sheath may be optionally flattened, tapered, or oval, among other configurations. The steerable portion of the sheath may include a cross-sectional profile in a plane generally transverse to the longitudinal axis that extends through the sheath between the proximal end and the distal end. The shape of a cross-sectional profile of the steerable portion of the sheath may include a major dimension and a minor dimension, wherein the minor dimension is shorter than the major dimension. The major dimension of the steerable portion is the longest dimension across the cross-sectional profile of the steerable portion. The minor dimension of the steerable portion is the shortest dimension across the cross-sectional profile of the steerable portion.

Further, the shape of the cross-sectional profile of the steerable portion of the sheath is preferably configured to, for example, reduce the likelihood of inflicting damage on a tubular structure to be ligated (e.g., avoiding injury to a pulmonary artery) when the steerable portion of the sheath may be, for example, manipulated near the tubular structure to be ligated and/or deflected around the tubular structure to be ligated.

The shape of the sheath and/or other indicators may also be used to provide feedback to a use regarding the orientation of the sheath (and, thus, the port or ports on the sheath). More particularly, for example, the flattened surfaces or other features of the sheath may allow the user to determine whether the sheath is oriented properly such that its deflection will cause it to curve around a tubular structure to be ligated rather than away from the tubular structure. The features other than shape of the sheath that may be used to provide feedback to a user regarding orientation of the sheath may include, but are not limited to color, surface texture, symbols, etc.

In one or more embodiments, a suture used in connection with the present invention may include a hollow suture that contains a stiffening element located therein. In some embodiments, only a portion of the stiffening element may be located within a hollow suture. A stiffening element may be provided within a hollow suture to provide support to the suture and/or to enhance placement of the suture.

A stiffening element may include a metallic and/or polymeric wires or other structures that may be positioned within a hollow suture. A stiffening element, according to the present disclosure, may be constructed from one or more of a wide variety of suitable materials such as polymers (e.g., shape memory polymers, etc.), metals (e.g., nitinol, stainless steel, etc.), composites, etc. Some potentially suitable stiffening element materials may include, e.g., but are not limited to, those mentioned in PCT Application Publication No. WO 2008/036408 (Friedman et al.). One having ordinary skill in the art will recognize other suitable stiffening element materials.

A ligation device according to the present disclosure may include a second lumen extending through the sheath towards the distal end of the sheath, wherein the second lumen is generally aligned with the longitudinal axis, and wherein the second lumen includes a second port proximate the distal end of the sheath, wherein the second port opens into the second lumen, and further wherein the second port is located in the steerable portion of the sheath. The ligation device may further include a second suture located within the second lumen, wherein the second suture can be advanced out of the second lumen through the second port.

In embodiments having more than one port, the diameters of the ports may be the same or different. In embodiments containing more than one port, the ports may be separated by any suitable distance, preferably 2 to 3 millimeters.

A ligation device that includes a sheath, two lumens, and two sutures may allow a tubular structure (e.g., an artery) to be simultaneously ligated on both sides of where the tubular structure will be cut. In other embodiments, the tubular structure may only be cut or severed on one side of the multiple (in this case two) ligation sites, with the multiple ligation sites potentially providing a more secure ligation of the tubular structure before and after the severing.

A ligation device according to the present disclosure may further include a suture fastener. A suture fastener may be a fastening device that can secure together, for example, two or more portions (e.g. ends or strands) of a suture. Suture fasteners and knot pushing devices may be used to, for example, ligate the tubular structure to be ligated. In one or more embodiments, the suture fastener may include a suture lock, clip, fastener, or any other suitable securement device. Examples of suture locking devices may be found in U.S. Patent Application Publication No. 2007/0270907 (Stokes et al.). A suture fastener according to the present disclosure may include a knot pushing device. Examples of knot pushing devices are provided in U.S. Pat. No. 5,759,189 (Ferragamo et al.), U.S. Pat. No. 5,769,863 (Garrison), and U.S. Pat. No. 6,132,439 (Kontos).

In one or more embodiments wherein the ligation device includes more than one suture, each suture may be fastened with a suture fastener (e.g., suture lock, fastener, clip, or other securement device) and/or a knot pusher may be used to tie each suture, thereby ligating the tubular structure to be ligated.

The present disclosure also provides methods of ligating a tubular structure to be ligated. In one or more embodiments, a ligation method may include providing a ligation device of the present disclosure, deflecting the steerable portion of the sheath to form a curve proximate the distal end of the sheath; advancing the suture out of the lumen through the port on the distal end of the sheath; removing the sheath from the suture; and ligating a tubular structure to be ligated with the suture.

It may be preferred to deflect the steerable portion sufficiently, such that a suture may be advanced out of the lumen through the port without contacting the tubular structure to be ligated with the advancing suture.

In one or more embodiments, advancing the suture out of the lumen through the port may optionally include grasping the suture and/or stiffening element with a grasping device to advance the suture and/or stiffening element through the port. A grasping device may be employed, for example, when the methods and/or devices of the present disclosure are employed in a thorascopic surgical procedure. The grasping device may be adapted to reach through a thorascopic passage to grasp the suture and/or the stiffening element in order to advance the suture and/or stiffening element out of the lumen through the port. A grasping tool may optionally include grasping forceps or a similar device. Examples of potentially suitable forceps may be found in U.S. Pat. No. 5,792,177 (Kaseda).

In one or more embodiments of the present disclosure, the tubular structure to be ligated may include a bodily fluid of an animal, preferably of a human or other mammal. Preferably, the tubular structure to be ligated may be a blood vessel or other vessel through which bodily fluids transport. More preferably, the tubular structure to be ligated may be an artery. Further, even more preferably, the tubular structure to be ligated may be a pulmonary artery.

Methods of the present disclosure may include using a ligation device that includes a second lumen extending through the sheath towards the distal end of the sheath, wherein the second lumen is generally aligned with the longitudinal axis, and wherein the second lumen includes a second port proximate the distal end of the sheath, wherein the second port opens into the second lumen, and further wherein the second port is located in the steerable portion of the sheath, and wherein the device further includes a second suture located within the second lumen, wherein the second suture can be advanced out of the second lumen through the second port. In one or more embodiments, the method may further include advancing the second suture out of the second lumen through the second port on the distal end of the sheath; removing the sheath from the second suture; and ligating the tubular structure to be ligated with the second suture.

In one or more embodiments of the present disclosure wherein the ligation device includes more than one suture, each suture may or may not independently include a hollow suture that may contain a stiffening element located therein and a method of using a ligation device that includes more than one suture may further include removing each stiffening element from each hollow suture.

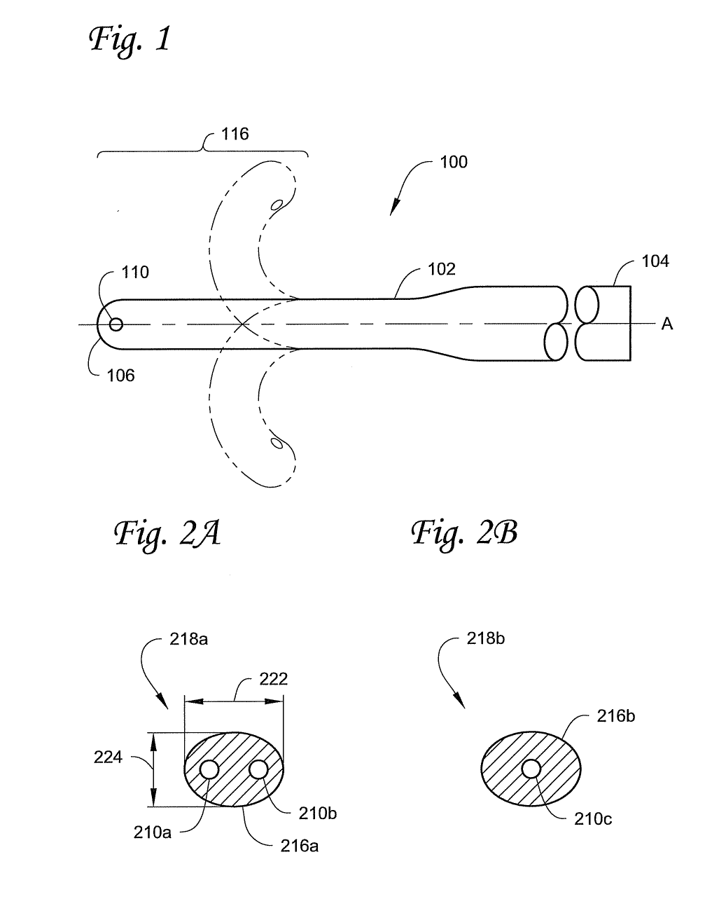

FIG. 1 shows one embodiment of a ligation device 100. The ligation device includes a sheath 102 that includes a distal end 106 and a proximal end 104, wherein a longitudinal axis (shown as dashed line A) extends through the sheath 102 between the proximal end 104 and the distal end 106.

A steerable portion of a sheath may be capable of deflecting in one or more than one direction. FIG. 1 shows a steerable portion 116 of the sheath 102. As shown in FIG. 1, the magnitude and/or direction of the deflection of the steerable portion 116 can vary.

Also depicted in FIG. 1 is port 110 located in the steerable portion 116 of the sheath 102.

FIGS. 2(a) and 2(b) each show an example of cross-sectional profiles 218a and 218b of a steerable portion 216a and 216b of a sheath, respectively.

FIG. 2(a) shows a cross-sectional profile 218a of a steerable portion 216a of a sheath that includes two lumens 210a and 210b. Also shown in FIG. 2(a) are the major dimension 222 and minor dimension 224 of the cross-sectional profile 218a of a steerable portion 216a of a sheath.

FIG. 2(b) shows a cross-sectional profile 218b of a steerable portion 216b of a sheath that includes one lumen 210c.

It can be seen in FIGS. 2(a) and 2(b) that the cross-sectional profiles 218a and 218b of the steerable portion 216a and 216b have major dimensions longer than the minor dimensions. The steerable portions 216a and 216b are shown as having an oval shape, although other flattened shapes may be used as discussed herein.

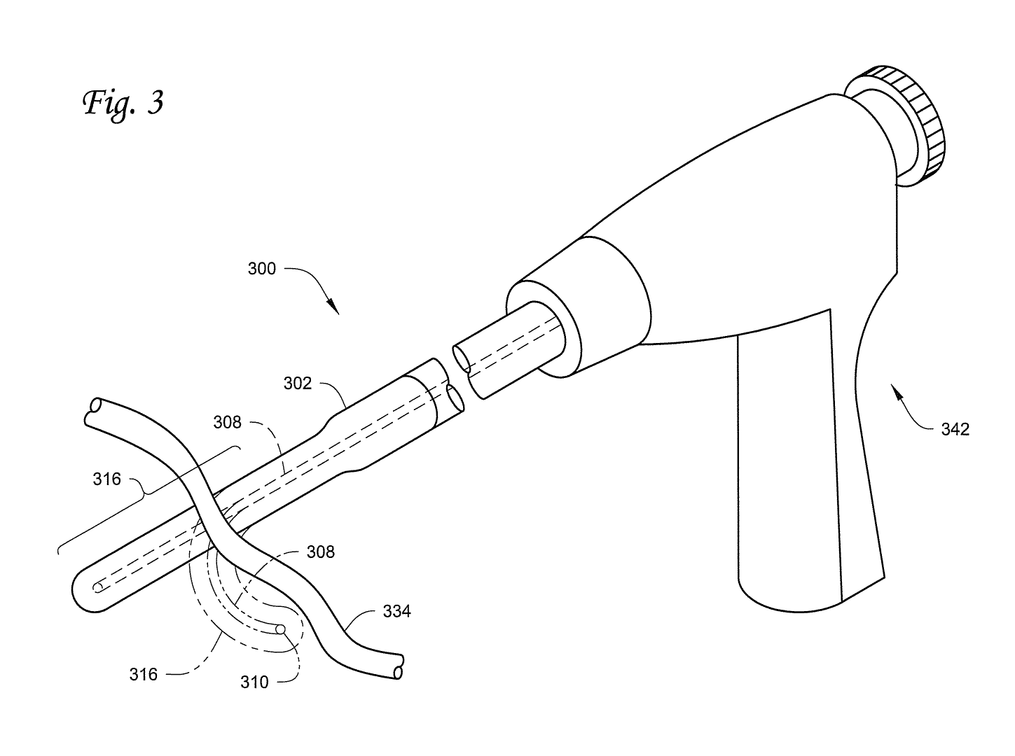

FIG. 3 is a schematic depicting the steerable portion 316 of a ligation device 300 being deflected around a tubular structure to be ligated 334. The steerable portion 316 of the ligation device includes a lumen 308 extending through the sheath 302. FIG. 3 also indicates a port 310 opening into the lumen 308.

The deflection of the steerable portion 316 of the ligation device 300 may be performed by manipulating controls on actuator 342. The deflection of the steerable portion 316 may form a curve proximate the distal end of the sheath 302

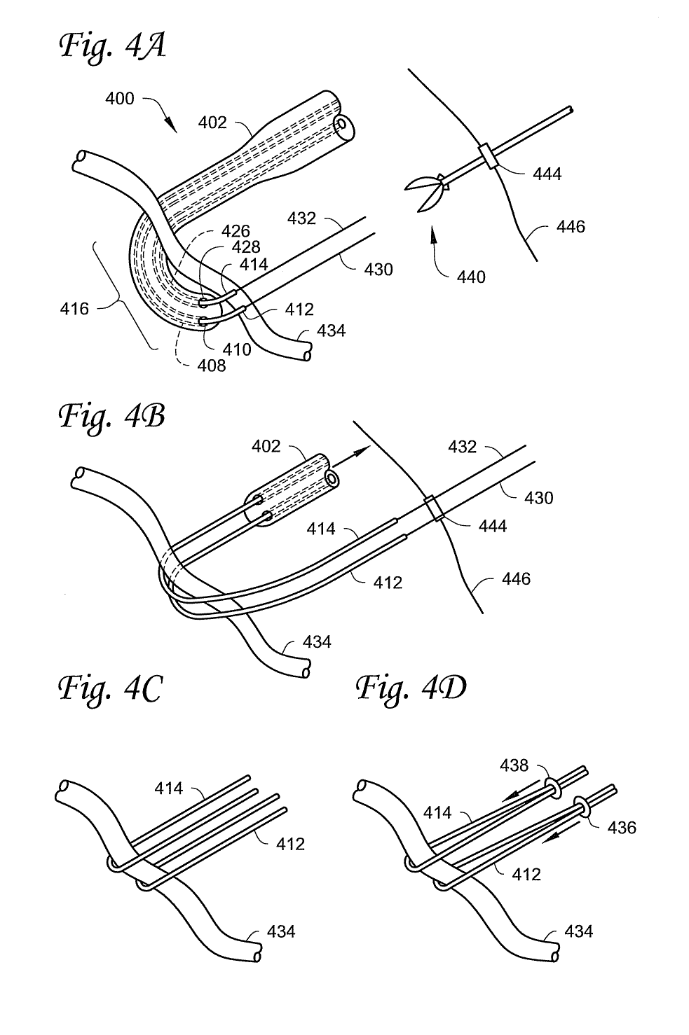

FIG. 4(a) depicts an embodiment of the present disclosure which includes a ligation device 400 having a sheath 402 having a steerable portion 416 at the distal end of the sheath 402, two ports 410 and 428 located in the steerable portion 416 and opening into two lumens 408 and 426. FIG. 4 shows two sutures 412 and 414 located within the two lumens 408 and 426, extending through the two ports 410 and 428. The sutures 412 and 414 are drawn as hollow sutures, each containing a stiffening element 430 and 432. The steerable portion 416 has been deflected around a tubular structure to be ligated 434.

FIG. 4(a) also shows a grasping device 440, which may be used to grasp the sutures 412 and 414 and/or the stiffening elements 430 and 432, after at least a portion of which has been advanced out of the lumens 408 and 426 through the ports 410 and 428. Grasping the sutures 412 and 414 and/or the stiffening elements 430 and 432 may allow advancing (e.g., by pulling) the sutures 412 and 414 out of the lumens 408 and 426 through the ports 410 and 428. The grasping tool may be optionally adapted to extend through a thorascopic port 444 in the skin 446 of an animal (e.g., a human).

FIG. 4(b) shows an embodiment wherein the sutures 412 and 414 and stiffening elements 430 and 432 have been advanced such that they may extend out of a thorascopic port 444 in the skin 446 of an animal (e.g., a human). FIG. 4(b) also indicates removing the sheath 402 from the sutures 412 and 414. Removing the sheath 402 from the sutures 412 and 414 may be performed such that the sutures remain in place around the tubular structure to be ligated 434.

After the sheath 402 is removed from the sutures 412 and 414 and the stiffening elements 430 and 432 are removed from the sutures 412 and 414, the sutures 412 and 414 remain in position around the tubular structure to be ligated 434, as shown in FIG. 4(c).

The sutures 412 and 414 may be used to ligate the tubular structure 434. In FIG. 4(d), the ligating is performed by advancing a suture fastener 436 and 438 on each suture 412 and 414 toward the tubular structure to be ligated 434. Any suitable suture fastener/lock constructions may be used to secure the sutures used in connection with the ligation devices described herein.

In one or more embodiments, deflecting the steerable portion of the sheath may form a curve proximate the distal end of the sheath, whereby the curve extends around at least a portion of a tubular structure to be ligated, as shown in FIGS. 3 and 4(a).

Methods and devices of the present disclosure may be employed during any suitable surgical procedures, including open surgical procedures, thorascopic surgical procedures, or other types of surgical procedures. Hence, in one or more embodiments, providing a ligation device as described herein for ligating a tubular structure may optionally include advancing the sheath through a thorascopic port to a position proximal the tubular structure to be ligated (e.g., a pulmonary artery).

In methods of the present disclosure, ligating the tubular structure to be ligated may optionally include forming a knot around the tubular structure to be ligated with the distal end and proximal end of the suture and pushing the knot (e.g., with a knot pusher) to the tubular structure to be ligated. Ligating the tubular structure to be ligated may optionally include applying a suture fastener to the suture to ligate the tubular structure.

One of ordinary skill in the art will recognize that the methods and devices of the present disclosure may include any suitable number (e.g., one, two, three, four, etc.) of lumens, and sutures in order to ligate one or more tubular structures to be ligated.

In one or more embodiments, a tubular structure may be ligated and severed. A tubular structure may be ligated on one side of ligation site and/or between sites of ligation. In embodiments in which multiple ligation sites are provided and the tubular structure is cut or severed on one side of the multiple ligation sites, the multiple ligation sites may potentially provide a more secure ligation of the tubular structure before and after the severing. Examples of devices and methods for ligating and severing blood vessels are provided in U.S. Pat. No. 6,679,892 (Guido et al.).

The words “preferred” and “preferably” as used herein refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a lumen may be used to refer to one, two, three or more lumens.

The term “and/or” means one or all of the listed elements or a combination of any two or more of the listed elements.

All references and publications cited herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A ligation device comprising:
a sheath comprising a distal end and a proximal end, wherein a longitudinal axis extends through the sheath between the proximal end and the distal end;
a lumen extending through the sheath towards the distal end of the sheath, wherein the lumen is generally aligned with the longitudinal axis, and wherein the lumen comprises a port proximate the distal end of the sheath, wherein the port opens into the lumen;
a suture located within the lumen, wherein the suture can be advanced out of the lumen through the port;
wherein the sheath comprises a steerable portion proximate the distal end, wherein the steerable portion can be deflected such that the steerable portion fauns a curve proximate the distal end of the sheath, and wherein the port is located within the steerable portion of the sheath.

2. A ligation device according to claim 1, wherein the steerable portion of the sheath is flattened such that the steerable portion of the sheath comprises a cross-sectional profile that includes a major dimension and a minor dimension.

3. A ligation device according to claim 1, wherein the suture comprises a hollow suture that contains a stiffening element located therein.

4. A ligation device according to claim 1, wherein the device further comprises:
a second lumen extending through the sheath towards the distal end of the sheath, wherein the second lumen is generally aligned with the longitudinal axis, and wherein the second lumen comprises a second port proximate the distal end of the sheath, wherein the second port opens into the second lumen, and further wherein the second port is located in the steerable portion of the sheath;
a second suture located within the second lumen, wherein the second suture can be advanced out of the second lumen through the second port.

5. A ligation device according to claim 4, wherein the second suture comprises a second hollow suture.

6. A ligation device according to claim 1, further comprising a suture fastener.

7. A ligation method comprising:
providing the ligation device of claim 1;
deflecting the steerable portion of the sheath to form a curve proximate the distal end of the sheath;
advancing the suture out of the lumen through the port on the distal end of the sheath;
removing the sheath from the suture; and
ligating a tubular structure to be ligated with the suture.

8. The ligation method of claim 7, wherein the device further comprises:
a second lumen extending through the sheath towards the distal end of the sheath, wherein the second lumen is generally aligned with the longitudinal axis, and wherein the second lumen comprises a second port proximate the distal end of the sheath, wherein the second port opens into the second lumen, and further wherein the second port is located in the steerable portion of the sheath;
a second suture located within the second lumen, wherein the second suture can be advanced out of the second lumen through the second port;
and the method further comprises:
advancing the second suture out of the second lumen through the second port on the distal end of the sheath;
removing the sheath from the second suture; and
ligating the tubular structure to be ligated with the second suture.

9. The ligation method of claim 8, wherein each suture optionally comprises a hollow suture that contains a stiffening element located therein and the method further comprises removing each stiffening element from each hollow suture.

10. A ligation device comprising:
a sheath comprising a distal end and a proximal end, wherein a longitudinal axis extends through the sheath between the proximal end and the distal end;
a first lumen extending through the sheath towards the distal end of the sheath, wherein the first lumen is generally aligned with the longitudinal axis, and wherein the first lumen comprises a first port proximate the distal end of the sheath, wherein the first port opens into the lumen;
a first suture located within the first lumen, wherein the first suture can be advanced out of the first lumen through the first port;
a second lumen extending through the sheath towards the distal end of the sheath, wherein the second lumen is generally aligned with the longitudinal axis, and wherein the second lumen comprises a second port proximate the distal end of the sheath, wherein the second port opens into the second lumen;
a second suture located within the second lumen, wherein the second suture can be advanced out of the second lumen through the second port;
wherein the sheath comprises a steerable portion proximate the distal end, wherein the steerable portion can be deflected such that the steerable portion forms a curve proximate the distal end of the sheath, wherein the steerable portion of the sheath is flattened such that the steerable portion of the sheath comprises a cross-sectional profile that includes a major dimension and a minor dimension, and wherein the first port and the second port are located within the steerable portion of the sheath.

11. A ligation device according to claim 10, wherein the first suture comprises a hollow suture that contains a stiffening element located therein.

12. A ligation device according to claim 11, wherein the second suture comprises a hollow suture that contains a stiffening element located therein.

13. A ligation device according to claim 10, further comprising a first suture fastener operably attached to the first suture.

14. A ligation device according to claim 13, further comprising a second suture fastener operably attached to the second suture.
